(19) United States
(12) Patent Application Publication
(10) Pub. No.: US 2008/0227972 A1
(43) Pub. Date: Sep. 18, 2008

(54) Decomposition method of cellulose and production method of glucose

(76) Inventor: Norishige Yamaguchi, Chiba-shi (JP)

Correspondence Address: BRUCE L. ADAMS, ESQ;ADAMS & WILKS, SUITE 1231, 17 BATTERY PLACE, NEW YORK, NY, 10004, US

(21) Appl. No.: 12/074,827

(22) Filed: Mar. 6, 2008

(30) Foreign Application Priority Data
Mar. 16, 2007 (JP) 2007-068943

(51) Int. Cl. C07H 3/02

(52) U.S. Cl. 536/124

(57) ABSTRACT

A method for decomposing cellulose to be contained in a cellulose raw material is provided. A pulverized cellulose based biomass is enclosed in a pressure closed vessel, and a sodium hydroxide aqueous solution having a concentration of 5%, pure water and 5 g of nickel oxyhydroxide obtained by solid-solving therein at least one kind of zinc, aluminum, magnesium, calcium, manganese, cobalt, copper and tin relative to nickel are added to prepare a catalytic reaction solution. Next, the catalytic reaction solution for decomposing cellulose by using nickel oxyhydroxide as a catalyst is subjected to a decomposition reaction of cellulose while stirring by using a stirring blade and heating at a temperature rising rate of 5° C./min. The reaction is carried out under autogenous pressure (saturated vapor pressure of water) in the reactor. After the temperature of the catalytic reaction solution has reached a prescribed temperature, the resulting catalytic reaction solution is heated for one hour and then cooled to room temperature at a rate of about 3° C./min.

### BACKGROUND OF THE INVENTION

1. Field of the Invention

The present invention relates to a method for decomposing cellulose by using nickel oxyhydroxide as a catalyst for a cellulose-containing cellulose raw material and a method for producing glucose.

2. Description of the Related Art

These days, a biomass is watched in grappling with carbon dioxide reduction (anti-global warming measure), construction of society with an environmentally-sound material cycle and so on. Examples of a vegetable biomass include cellulose, and this cellulose has various applications, for example, as a raw material for useful derivatives such as cellulose esters and cellulose ethers or a raw material for ethanol.

Cellulose is composed of a cell wall of vegetable cell and a fiber as major components and is the most common hydrocarbon among organic materials produced in the natural world. The cellulose is a natural polymer in which a number of β-glucose molecules are linearly polymerized by a glycoside linkage. In the natural state, the cellulose frequently exists linked with hemicelluloses or lignin.

In order to utilize cellulose, it is necessary to decompose the cellulose. As a decomposition method thereof, an acid hydrolysis method using sulfuric acid or hydrochloric acid and an enzymatic hydrolysis method using cellulase as an enzyme are studied.

Concretely, as a method of using pressurized hot water, a method for bringing a cellulose powder into contact with pressurized hot water heated at 200 to 300° C. to hydrolyze the cellulose powder is studied (see, for example, Patent Document 1 described below). A method for hydrolyzing a vegetable biomass with pressurized hot water which has been pressurized to a saturated vapor pressure or higher at 140° C. to 230° C. to extract cellulose and decomposing the cellulose with a nickel based catalyst in an atmosphere heated at 380 to 420° C. (see, for example, Patent Document 2 described below) and the like are also proposed.

A method for depolymerizing a cellulose ether that flocculates in hot water by hydrolysis with a mineral acid or an organic acid is also reported (see, for example, Patent Document 3 described below).

Besides, as a method of using a solid catalyst, a method for treating a reaction solution containing cellulose and the catalyst at 125 to 250° C. by using active carbon having an acidic functional group or a basic functional group in a molecule thereof, etc. (see, for example, Patent Document 4 described below) and the like are also proposed.

Patent Document 1: JP-A-10-327900 (page 1)

Patent Document 2: JP-A-2002-59118 (page 1)

Patent Document 3: JP-T-2003-508597 (page 1)

Patent Document 4: JP-A-2006-129735 (page 1)

However, cellulose is very stable and hardly decomposable, and therefore, its industrial utilization is disturbed. That is, the method of using cellulase involves a defect that the rate of hydrolysis is extremely slow because of a firm crystal structure of cellulose.

Also, in the methods of using pressurized hot water as described in Patent Documents 1 and 2, the hydrolysis cannot be efficiently carried out because the progress of the reaction is slow. Furthermore, since it is necessary to pressurize hot water, a pressurization device becomes necessary, and the apparatus as a whole becomes large in size. Thus, these methods are not efficient.

Also, the hydrolysis using a chemical such as acids as described in Patent Document 3 is high in costs. Furthermore, since this chemical has stimulativeness, a problem that a load against the environment is large is generated.

Furthermore, the method of using a solid catalyst as described in Patent Document 4 uses the solid catalyst, and therefore, the hydrolysis decomposition step is not complicated. However, the decomposition reaction temperature is high as 125 to 250° C., and a problem that the energy efficiency for achieving the production is low is involved.

In order to solve the foregoing problems, the invention has been made, and an object thereof is to provide a method for decomposing cellulose to be contained a cellulose raw material.

### SUMMARY OF THE INVENTION

In order to solve the foregoing problems, a first aspect of the invention is concerned with a method for decomposing cellulose by heating a reaction solution composed of a cellulose-containing cellulose raw material and an alkaline aqueous solution at a prescribed temperature, wherein nickel oxyhydroxide is added as a catalyst for promoting a decomposition reaction of cellulose to the reaction solution.

A second aspect of the invention is concerned with the method for decomposing cellulose as set forth in the first aspect of the invention, wherein the nickel oxyhydroxide solid-solves therein at least one kind of zinc, aluminum, magnesium, calcium, manganese, cobalt, copper and tin.

A third aspect of the invention is concerned with the method for decomposing cellulose as set forth in the first aspect of the invention, wherein the cellulose raw material contains chemically modified cellulose.

A fourth aspect of the invention is concerned with the method for decomposing cellulose as set forth in the second aspect of the invention, wherein the cellulose raw material contains chemically modified cellulose.

A fifth aspect of the invention is concerned with the method for decomposing cellulose as set forth in any one of the first to fourth aspects of the invention, wherein the prescribed temperature for heating is 80° C. or higher and not higher than 130° C.

A sixth aspect of the invention is concerned with a method for producing glucose by heating a reaction solution composed of a cellulose-containing cellulose raw material and an alkaline aqueous solution at a prescribed temperature to decompose cellulose, wherein nickel oxyhydroxide is added as a catalyst for promoting a decomposition reaction of cellulose to the reaction solution.

The invention of this application is based on knowledge that when nickel oxyhydroxide having an oxy structure is used as a catalyst in a decomposition reaction of cellulose, cellulose is efficiently decomposed, which knowledge has been first clarified by the present inventor. According to this, it is possible to decompose cellulose cheaply and efficiently at low energy, thereby obtaining glucose as a product.

A catalytic reaction is initiated due to the matter that a reactant molecule is coordinated with or adsorbed on a catalyst and as compared with a homogeneous reaction of the same molecule, remarkably reduces activation energy due to the matter that the reactant molecule coordinated with or adsorbed on the catalyst weakens or dissociates an intramolecular linkage, thereby increasing the rate of reaction. On the occasion that the reactant molecule is coordinated with or adsorbed on the catalyst, the surface area or surface charge that the catalyst has plays an important role. Therefore, by using nickel oxyhydroxide (NiOOH) having an O═ group and an OH—group in a molecule thereof as the catalyst, it is possible to enhance the efficiency of coordination or adsorption of the reactant molecule on the catalyst by a hydrogen bond.

According to this, the decomposition of cellulose can be achieved by simple procedures of heating the reaction solution containing a cellulose raw material and a nickel oxyhydroxide catalyst. In consequence, cellulose can be very simply and cheaply decomposed with good efficiency, and a large-scale reaction apparatus is not required.

According to the method for decomposing cellulose by using nickel oxyhydroxide as a catalyst, in the heat treatment, the nickel oxyhydroxide efficiently adsorbs cellulose in the cellulose raw material on an O═ group and an OH— group in a molecule thereof via a hydrogen bond, thereby promoting the decomposition of cellulose. In consequence, by using chemically modified cellulose, glucose as a hydrolyzate of cellulose can be rapidly and efficiently obtained.

Also, the decomposition method of cellulose and the production method of glucose according to the invention are characterized in that the prescribed temperature for decomposing cellulose by using nickel oxyhydroxide as a catalyst is 80° C. or higher and not higher than 130° C. Here, when the reaction temperature is lower than 80° C., a delay of the decomposition reaction is brought. On the other hand, when the reaction temperature exceeds 130° C., the energy for decomposing cellulose is consumed corresponding thereto, and the efficiency to the energy that a decomposition reaction product of cellulose such as glucose has is reduced. In consequence, by decomposing cellulose within the temperature range at which cellulose is decomposed by using nickel oxyhydroxide as a catalyst, not only the decomposition can be rapidly achieved, but a decomposition reaction product of cellulose can be efficiently produced.

Cellulose contained in the cellulose raw material can be efficiently decomposed, and glucose as a product can be efficiently obtained. Also, since the method according to the invention is very simple, it is possible to cheaply decompose cellulose.

### BRIEF DESCRIPTION OF THE DRAWING

FIG. 1 is an explanatory view of Examples and Comparative Examples of the invention.

### DETAILED DESCRIPTION OF THE INVENTION

An embodiment which embodies the invention is hereunder explained with reference to the decomposition method of cellulose. The production method of glucose according to the invention employs the decomposition method of cellulose according to the invention. In consequence, the explanation of the production method of glucose according to the invention is common to the explanation of the decomposition method of cellulose according to the invention.

The decomposition method of cellulose includes the steps of preparing a catalytic reaction solution composed of a cellulose-containing cellulose raw material, an alkaline aqueous solution and nickel oxyhydroxide capable of catalyzing a decomposition reaction of cellulose; and heating this solution at a prescribed temperature.

The cellulose content of the cellulose raw material which is used in this embodiment is not particularly limited and may be one such that cellulose to be contained therein can be dispersed in the catalytic reaction solution. Though the shape of the cellulose raw material is not particularly limited, a powdered shape is preferable because it is easy to disperse it in water.

In this embodiment, nickel oxyhydroxide which is used as the catalyst solid-solves therein at least one kind of cobalt and copper.

It is preferable that the reaction solution for decomposing cellulose by using nickel oxyhydroxide as a catalyst contains readily decomposable, chemically modified cellulose having a high content of cellulose.

The heat treatment of the invention is a step of heating a catalytic reaction solution for decomposing cellulose by using nickel oxyhydroxide as a catalyst at a prescribed temperature, thereby making the nickel oxyhydroxide act on cellulose. This heating method is not particularly limited and can be carried by employing a conventionally known method. For example, heating can be achieved by using a commercially available autoclave.

Though the reaction temperature is not particularly limited, when it is 80° C. or higher, an effective rate of reaction can be secured. From the viewpoint of simplicity of a heating device, the reaction temperature is preferably not higher than 130° C. By making the reaction temperature fall within the range wherein nickel oxyhydroxide functions as a catalyst to decompose cellulose, it is possible to efficiently make the nickel oxyhydroxide catalyst act on cellulose. When the reaction temperature is lower than 80° C., the decomposition reaction is delayed, whereas when it exceeds 130° C., the energy for producing glucose is consumed corresponding thereto, and the production efficiency against the energy that glucose has is reduced; and therefore, such is not preferable.

Though the temperature rising rate of the catalytic reaction solution is not particularly limited, from the viewpoint of inhibiting excessive decomposition of the product, it is preferable that the temperature rising rate is relatively low as not more than 10° C./min. For example, in the case of a heating device utilizing solar heat, etc., such a temperature rising rate can be realized.

Though the cooling rate of the catalytic reaction solution is not particularly limited, from the viewpoint of simplicity of a cooling device, it is preferable that the cooling rate is relatively low as not more than 10° C./min as in, for example, natural cooling or water cooling.

The reaction time is not particularly limited. The catalytic reaction solution may be cooled immediately after it has reached a set reaction temperature or may be held at a set reaction temperature for an arbitrary time.

Though the pressure at the time of reaction is not particularly limited, for example, it is preferable that the reaction is carried out under autogenous pressure (saturated vapor pressure of water) in a reactor or a higher pressure. However, for the purpose of keeping the reaction condition constant, it is preferable that the pressure is kept constant. In consequence, it is preferable that the reaction is carried out in a pressure closed vessel.

In the light of the above, in the decomposition method of cellulose according to the invention, in the heating step, cellulose is decomposed by using a nickel oxyhydroxide catalyst, thereby producing glucose. According to this, the decomposition of cellulose contained in the cellulose raw material can be promoted.

The invention is not limited to the respective configurations as described previously; various changes and modifications can be made within the scope of the appended claims; and embodiments obtainable by properly combining technical measures as disclosed respectively in different embodiments are also included in the technical scope of the invention.

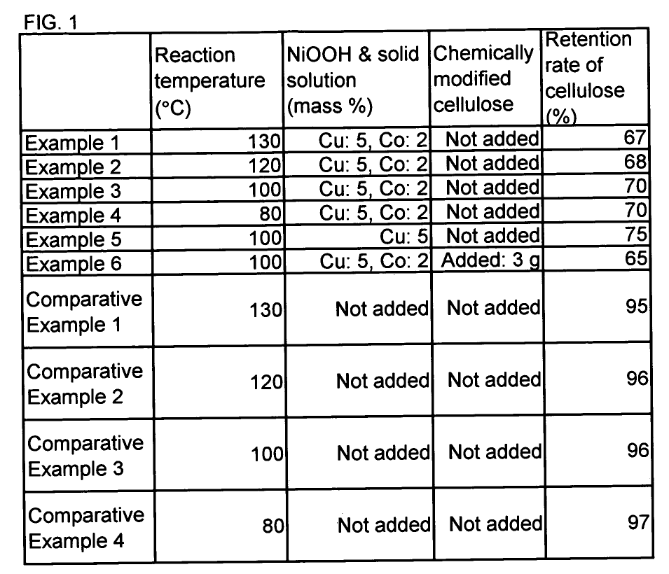

The invention is specifically described below with reference to the following Examples. As described later, these Examples were carried out under the conditions as described in an evaluation table as shown in FIG. 1, but it should not be construed that the invention is limited thereto.

### EXAMPLES

10.0 g of a pulverized cellulose based biomass (manufactured by Aldrich) was enclosed in a pressure closed vessel, and 50 g of a sodium hydroxide aqueous solution having a concentration of 5%, 600 g of pure water and 5 g of nickel oxyhydroxide obtained by solid-solving therein cobalt or copper relative to nickel were added to prepare a catalytic reaction solution. Next, the catalytic reaction solution for decomposing cellulose by using nickel oxyhydroxide as a catalyst was subjected to a decomposition reaction of cellulose while stirring by using a stirring blade and heating at a temperature rising rate of 5° C./min. The reaction was carried out under autogenous pressure (saturated vapor pressure of water) in the reactor. After the temperature of the catalytic reaction solution had reached a prescribed temperature as described in the evaluation table as shown in FIG. 1, the resulting catalytic reaction solution was heated for one hour and then cooled to room temperature at a rate of about 3° C./min. The “room temperature” as referred to herein means a range of usual room temperature (15 to 25° C.).

After cooling, the product within the vessel was recovered, and the amount of residual cellulose after completion of the reaction was calculated. With respect to the amount of residual cellulose, the catalytic reaction solution after completion of the reaction for decomposing cellulose by using nickel oxyhydroxide as a catalyst was cooled and then filtered through a filtration filter made of polyethersulfone; and an insoluble matter was washed several times with pure water and then dried at 105±5° C. for 2 hours or more, followed by weighing. With respect to the calculation method of the mass of residual cellulose, a mass obtained by subtracting previously measured masses of the filtration filter and nickel oxyhydroxide, etc. from the above-weighed mass was designated as “mass of residual cellulose”.

Then, a retention rate of cellulose in the case of carrying out the decomposition reaction upon addition of nickel oxyhydroxide was determined as a mass ratio (mass %) of cellulose to the mass of solids contained in the catalytic reaction solution after completion of the reaction at every reaction temperature. The evaluation results are shown in the evaluation table as shown in FIG. 1.

### Example 1

The decomposition of cellulose was carried out in the same manner under the foregoing condition, except that the reaction temperature was 130° C. and that the solid solution of nickel oxyhydroxide contained 5 mass % of copper and 2 mass % of cobalt relative to nickel.

### Example 2

The decomposition of cellulose was carried out in the same manner under the foregoing condition, except that the reaction temperature was 120° C. and that the solid solution of nickel oxyhydroxide contained 5 mass % of copper and 2 mass % of cobalt relative to nickel.

### Example 3

The decomposition of cellulose was carried out in the same manner under the foregoing condition, except that the reaction temperature was 100° C. and that the solid solution of nickel oxyhydroxide contained 5 mass % of copper and 2 mass % of cobalt relative to nickel.

### Example 4

The decomposition of cellulose was carried out in the same manner under the foregoing condition, except that the reaction temperature was 80° C. and that the solid solution of nickel oxyhydroxide-contained 5 mass % of copper and 2 mass % of cobalt relative to nickel.

### Example 5

The decomposition of cellulose was carried out in the same manner as in Example 3, except that the solid solution of nickel oxyhydroxide contained 5 mass % of copper relative to nickel.

### Example 6

The decomposition of cellulose was carried out in the same manner as in Example 3, except that the reaction solution for decomposing cellulose contained 3 g of chemically modified cellulose (including viscose, etc.).

### Comparative Example 1

The decomposition of cellulose was carried out in the same manner as in Example 1, except that the nickel oxyhydroxide was not added to the reaction solution for decomposing cellulose.

### Comparative Example 2

The decomposition of cellulose was carried out in the same manner as in Example 2, except that the nickel oxyhydroxide was not added to the reaction solution for decomposing cellulose.

### Comparative Example 3

The decomposition of cellulose was carried out in the same manner as in Example 3, except that the nickel oxyhydroxide was not added to the reaction solution for decomposing cellulose.

### Comparative Example 4

The decomposition of cellulose was carried out in the same manner as in Example 4, except that the nickel oxyhydroxide was not added to the reaction solution for decomposing cellulose.

### (Evaluation Results)

In comparing the results of Examples 1 to 4 and Comparative Examples 1 to 4 of the evaluation table as shown in FIG. 1, at the reaction temperature of 80 to 130° C., the case of not adding nickel oxyhydroxide reveals the results that the retention rate of cellulose is 95 to 97%, whereas the case of adding nickel oxyhydroxide reveals the results that the retention rate of cellulose is 67 to 70%. It is noted from this matter that the decomposition of cellulose is rapidly accelerated due to the presence of nickel oxyhydroxide. It is judged that in the decomposition reaction of cellulose, nickel oxyhydroxide efficiently works as a catalyst.

Next, in comparing the results of Example 3 and Example 5 of the evaluation table as shown in FIG. 1, the case where the solid solution of nickel oxyhydroxide contains 5 mass % of copper and 2 mass % of cobalt relative to nickel reveals the results that the retention rate of cellulose is 70%, whereas the case where the solid solution of nickel oxyhydroxide contains 5 mass % of copper relative to nickel reveals the results that the retention rate of cellulose is 75%. It is estimated from this matter that the catalytic ability in the decomposition of cellulose varies with the kind of a metal to be solid-solved in nickel oxyhydroxide, etc. It is estimated that the surface charge of nickel oxyhydroxide varies with the kind of a metal to be solid-solved, whereby an efficiency of the reactant molecule for coordination or adsorption on the catalyst is enhanced.

In comparing the results of Example 3 and Example 6 of the evaluation table as shown in FIG. 1, the case of containing chemically modified cellulose in the reaction solution reveals the results that the retention rate of cellulose is 65%, whereas the case of not containing chemically modified cellulose in the reaction solution reveals the results that the retention rate of cellulose is 70%. It is noted from this matter that by containing chemically modified cellulose in the reaction solution, the decomposition reaction of cellulose is promoted. It is estimated that because of the presence of glucose or the like as formed by the decomposition of cellulose between cellule molecules, the dissociation of a hydrogen bond between the molecules is promoted, whereby the decomposition reaction of cellulose is promoted.

According to this embodiment, the following effects can be obtained.

According to the foregoing embodiment, it has first become clear that cellulose can be efficiently decomposed by using nickel oxyhydroxide having an O═ group and an OH— group in a molecule thereof, and the decomposition of cellulose can be rapidly achieved. In consequence, it is possible to solve the problem that the progress of the reaction is so slow that the hydrolysis cannot be efficiently achieved as involved in the method of using pressurized hot water. Also, the reaction can be carried out through very simple steps, and a simple reaction device is enough to achieve the reaction; and therefore, it is possible to decompose cellulose easily and cheaply. As a result, it is possible to efficiently obtain glucose which is a decomposition product of cellulose.

The foregoing respective embodiments may be changed as follows.

In the foregoing Examples, the pulverized cellulose based biomass was used as the cellulose raw material. This cellulose raw material is not particularly limited so far as it contains cellulose. For example, a vegetable biomass, that is, organic materials including broad-leaved trees (for example, Japanese chinquapin), bamboo, conifers (for example, cedar), kenaf, waste limbers of furniture, rice straw, wheat straw and chaff can be used. Chemically modified cellulose separated from woods or the like can also be used.

In the foregoing Examples, in the case of decomposing cellulose by using nickel oxyhydroxide as a catalyst, the heat treatment was carried out. This heat treatment can be carried out while stirring the catalytic reaction solution. The stirring method is not particularly limited, and a conventionally known method can be employed. For example, the stirring may be achieved by using a known stirrer bar. Since the contact frequency between the nickel oxyhydroxide catalyst and cellulose can be increased by stirring the catalytic reaction solution, it is possible to enhance the efficiency of the cellulose hydrolysis reaction.

In the foregoing Examples, the decomposition method of cellulose was described. In this decomposition method of cellulose, it is also possible to include a recovery step of glucose and a purification step of glucose in addition to the step of decomposing cellulose by using nickel oxyhydroxide as a catalyst. The recovery step of glucose is not particularly limited. For example, a known method such as gel filtration and a method of using an ion exchange resin can be properly utilized. Examples of the purification step of glucose include operations such as recrystallization.

In the foregoing Examples, cobalt or copper was solid-solved in nickel oxyhydroxide. In that case, it is possible to use solid solution-free nickel oxyhydroxide as the catalyst.

Also, it is preferable that nickel oxyhydroxide solid-solves therein not only cobalt and copper but at least one kind of zinc, aluminum, magnesium, calcium, manganese and tin. By using such a solid solution, it is possible to change the polarization within nickel oxyhydroxide to promote the decomposition of cellulose.

In the foregoing Examples, there is a possibility that the decomposition reaction can be promoted by adding carbon such as active carbon to nickel oxyhydroxide.

The invention enables one to promote the utilization of cellulose as a raw material of glucose and is able to be utilized in a field where the utilization of glucose as a raw material of ethanol or the like can be thought, for example, an energy field, a food field and a chemical field.

What is claimed is:

1. A method for decomposing cellulose, comprising:
heating a reaction solution composed of a cellulose-containing cellulose raw material and an alkaline aqueous solution at a prescribed temperature; and
adding nickel oxyhydroxide as a catalyst for promoting a decomposition reaction of cellulose to the reaction solution.

2. A method for decomposing cellulose according to claim 1; wherein the nickel oxyhydroxide solid-solves therein at least one kind of zinc, aluminum, magnesium, calcium, manganese, cobalt, copper and tin.

3. A method for decomposing cellulose according to claim 2; wherein the cellulose raw material contains chemically modified cellulose.

4. A method for decomposing cellulose according to claim 1; wherein the cellulose raw material contains chemically modified cellulose.

5. A method for decomposing cellulose according to claim 1; wherein the prescribed temperature for heating is 80° C. or higher and not higher than 130° C.

6. A method for producing glucose, comprising:
heating a reaction solution composed of a cellulose-containing cellulose raw material and an alkaline aqueous solution at a prescribed temperature to decompose cellulose; and
adding nickel oxyhydroxide as a catalyst for promoting a decomposition reaction of cellulose to the reaction solution.
